(19) United States
(12) Patent Application Publication
(10) Pub. No.: US 2024/0010890 A1
(43) Pub. Date: Jan. 11, 2024

(54) FLUX-COMPATIBLE EPOXY-PHENOLIC ADHESIVE COMPOSITIONS FOR LOW GAP UNDERFILL APPLICATIONS

(71) Applicant: Henkel AG & Co. KGaA, Duesseldorf (DE)

(72) Inventors: Laxmisha M. Sridhar, Monmouth Junction, NJ (US); Zhan Hang Yang, Rocky Hill, CT (US)

(21) Appl. No.: 18/372,206

(22) Filed: Sep. 25, 2023

Related U.S. Application Data
(63) Continuation of application No. PCT/US2021/045140, filed on Aug. 9, 2021.

(30) Foreign Application Priority Data
Mar. 30, 2021 (TW) 110111505

(51) Int. Cl. C09J 163/10; C09J 11/06

(52) U.S. Cl. CPC C09J 163/10; C09J 11/06

(57) ABSTRACT

Provided are flux-compatible epoxy-phenol adhesive compositions useful as a low gap underfill and novel phenols useful therein. The flux-compatible epoxy-phenol adhesive compositions include an epoxy component including an epoxy compound having a cycloaliphatic, alicyclic or mixed cycloaliphatic-aromatic backbone, a multifunctional phenolic component, and a catalyst. The flux-compatible compositions are useful as an underfilling sealant which (1) rapidly fills the underfill space in a semiconductor device, such as a flip chip assembly, (2) enables the device to be securely connected to a circuit board by short-time heat curing and with good productivity, and (3) demonstrates excellent solder reflow resistance.

### FIELD

The present disclosure relates to flux compatible epoxy-phenolic adhesive compositions for low-gap underfill applications, and to novel phenols useful therein.

### BRIEF DISCUSSION OF RELATED TECHNOLOGY

The following discussion is provided merely to aid the reader in understanding the disclosure and is not admitted to describe or constitute prior art.

In recent years, the popularity of smaller-sized electronic appliances has made desirable size reduction of semiconductor devices. As a result, chip packages are becoming reduced in size to substantially that of the bare die itself. The trend of new package design to have more functions, finer pitch, a low gap, a thinner package, and extended downstream market not only comes with higher reliability requirements, but also has created many new challenges which did not exist for previous generations of underfill technology.

The flip-chip method of attaching an integrated circuit to an organic substrate board uses a series of metal solder bumps on the integrated circuit which form interconnections with the metal bond sites on the board. The active side of the integrated circuit is flipped upside down in order to make contact between the bumps on the integrated circuit and the metal bond sites on the substrate. An organic soldering flux is used to remove metal oxides and promote wetting of the solder when the assembly is heated above the melting temperature of the solder. This process of attaching an integrated circuit to a substrate is referred to as reflow soldering. The purpose of the flux is to clean the surface of the metals to improve electrical connection. The solder or lower melting alloy may comprise the metal bond sites on the substrate, the bumps on the integrated circuit, or both, depending on the materials selected. The higher melting alloy may also similarly be present in lead-free solder. Similarly, the higher melting alloy may be present in lead-free solder driven mainly by environmental concerns.

With small gap underfill, the residue from the flux is difficult to remove from the narrow gap. Thus, no-clean fluxes in which flux residues are not removed from the board after the solder reflow process are the flux choice for most flip-chip applications. These no-clean fluxes may be dispensed onto the metal bond sites on the board prior to chip placement. In order to maintain alignment of the chip to the board prior to the reflow process, a tacky flux may be applied to the bumps on the chip. The integrated circuit containing solder bumps is dipped into the flux to a set depth to apply a desired amount of tacky flux to only the surface of the bumps. The chip is then aligned and placed onto the substrate so that the flux-coated bumps contact the appropriate metal bond sites of the substrate. The tacky flux is formulated to contain a higher solids content, which aids in the adhesion of the chip to the substrate prior to reflow. The tacky flux thus acts as a temporary glue to hold the chip in proper alignment during placement of the assembly into the reflow oven. The tacky fluxes commonly used are the solder paste flux vehicles used in no-clean surface mount processes.

Although the formulations of no-clean solder paste flux vehicles vary, a typical composition contains 50% rosin, 40% solvent, 5-8% thickeners, and 2-5% flux activators (such as organic acids and amines). While most of the solvent of the flux boils off during the reflow process, the rosin ester and other nonvolatile residues of the solder paste constituents remain.

After solder reflow to attach the integrated circuit to the substrate, the gap between the integrated circuit and the organic substrate in a flip-chip assembly is filled with an underfill encapsulant or adhesive by capillary action. The purpose of the encapsulant is to relieve the thermomechanical stresses on the solder interconnections that are caused by the difference in thermal expansion coefficients between the silicon integrated chip (coefficient of thermal expansion (CTE)=2.5 ppm/° C.) and the organic substrate (CTE=15-20 ppm/° C.).

Typical underfill encapsulants used in flip-chip assemblies are composed of epoxy resins, curing agents, and inorganic fillers to yield a crosslinked thermosetting polymer when cured. The properties of the cured polymer, such as the CTE and elastic modulus, help relieve the thermomechanical stress on the solder joints during use, which is tested by thermal cycling testing. A typical thermal cycle test involves repeated exposure of the flip-chip assembly to two different liquids at −55° C. and 125° C. with a ten minute dwell time at each temperature. Thus, the overall purpose of the underfill encapsulant is to enhance the operational life and reliability of a flip-chip assembly by relieving the thermomechanical stress on the solder joints.

Several process and material property characteristics dictate the material selection of the underfill encapsulant. First, the epoxy underfill encapsulant should flow quickly under the chip during production. The viscosity, surface tension, and particle size distributions of the encapsulant can be optimized to achieve efficient flow under the chip during the encapsulation step. To further reduce the underfill time, the substrate may be heated in order to reduce the viscosity of the uncured encapsulant and enhance the flow speed of the material. It is common to heat the surface of the substrate board to 70° C. prior to dispensing the encapsulant in order to achieve this effect. Second, the epoxy underfill encapsulant should cure relatively quickly. Typical underfill encapsulants are epoxy formulations designed to cure, i.e. form irreversibly cross-linked structures, at temperatures of 130-170° C. Finally, the epoxy underfill encapsulant should adhere strongly to both the chip and substrate during thermal cycling tests. If the epoxy pulls away, or delaminates, from either the chip or substrate surface, proper stress relief on the interconnections will not be achieved.

The interaction between the no-clean flux residue and the epoxy underfill encapsulant is important to achieving maximum adhesion and desirable flip-chip reliability. Typical solder paste flux compositions used as tacky fluxes for the flip-chip process contain rosin or a similar resin. After the reflow soldering of the integrated circuit to the substrate, a residue of rosin and other nonvolatile organic constituents of the flux remain on the substrate. Although these no-clean residues are benign to the assembly in terms of their corrosivity, these residues have been known to cause voiding and solder extrusion, adversely affecting the adhesion and electrical integrity of the device. This result may lead to early delamination from the chip surface due to the poor adhesion of the underfill encapsulant. This delamination of the encapsulant from the chip can be detected and measured using scanning acoustic microscopy (SAM), which allows detection of the presence of voids between the surface of the chip and the epoxy underfill.

Thus, flux compatibility with the underfill encapsulant is an important criterion for underflow process performance.

A flux-compatible underfill adhesive that shows stable Tg when subjected to repeat solder reflow conditions and has low moisture absorption would be highly desirable. Maintaining stable Tg is important for good adhesion at higher temperature.

### SUMMARY

The present disclosure provides flux-compatible compositions useful as an underfilling sealant which (1) rapidly fills the underfill space in a semiconductor device, such as a flip chip assembly, (2) enables the device to be securely connected to a circuit board by short-time heat curing and with good productivity, and (3) demonstrates excellent thermal cycle properties. The compositions comprise an epoxy resin component, a phenolic component, and a catalyst. The present disclosure also provides novel phenols useful in the compositions.

Using the compositions of the present disclosure, semiconductor devices, such as flip chip assemblies, may be (1) assembled quickly and without production line down time because of improved cure speed and extended useful working life, and (2) securely connected to a circuit board by short-time heat curing of the composition, with the resulting mounted structure demonstrating excellent heat shock properties or thermal cycle properties.

Thus the present disclosure provides a flux-compatible epoxy-phenol adhesive composition usual for low gap underfill applications. The composition includes:
an epoxy component comprising an epoxy compound having a cycloaliphatic, alicyclic or mixed cycloaliphatic-aromatic, alicyclic-aromatic backbone;
a phenolic component, such as multifunctional phenol; and
a catalyst.

The epoxy component may be an epoxy compound having a cycloaliphatic, alicyclic, mixed cycloaliphatic-aromatic or alicyclic-aromatic backbone.

The phenolic component may be for instance a multifunctional phenol represented by the general structures 1, II, III and/or IV:

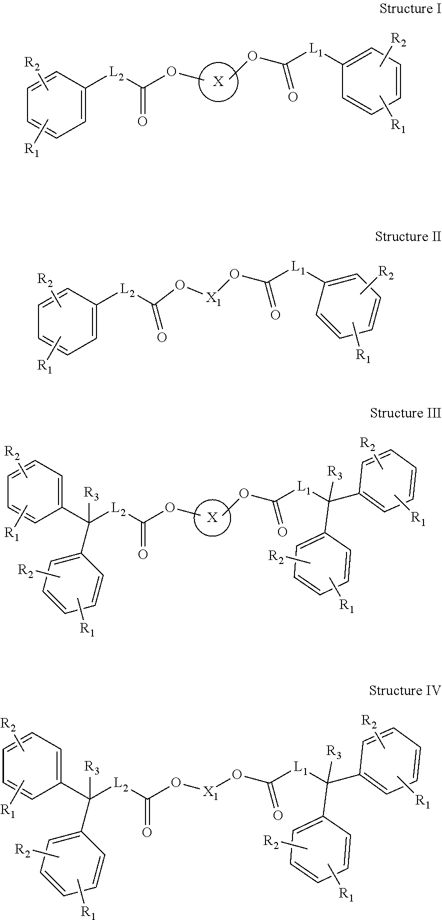

wherein X is a monocyclic, bicyclic or polycyclic ring structure that is cycloaliphatic, alicyclic or mixed cycloaliphatic-aromatic optionally with aliphatic side chains and the oxygen of the ester group can be connected directly to the ring or to the aliphatic side chain;
X₁ is alkylene or branched alkylene optionally comprising heteroatoms O or S;
R₃ is H, alkyl or cycloalkyl;
L₁ and L₂ are independently selected from a covalent bond, alkylene, branched alkylene, and cycloalkylene optionally comprising heteroatoms O or S;
R₁ and R₂ are H, methyl or OH with the proviso that at least one of R₁ or R₂ in each ring is OH; and
the ester group present in structures I, II, III and IV can be a primary or secondary ester group.
Or, the phenol component may be selected from structures V, VI and/or VII:

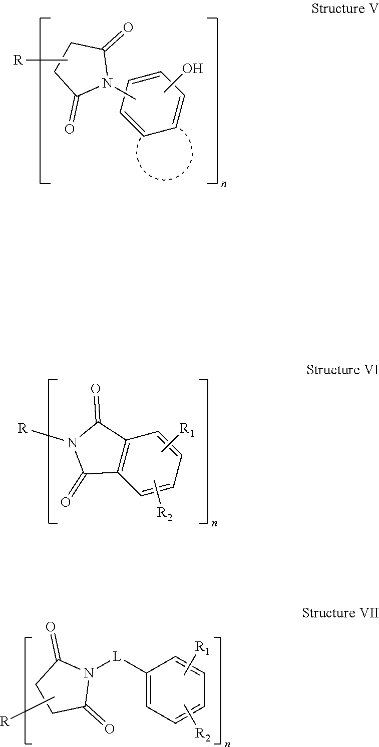

wherein R is an aliphatic, cycloaliphatic, alicyclic, mixed aromatic-cycloaliphatic or polymer backbone; in addition, R can be a fused ring in Structures V and VII;
R₁ and R₂ are H, alkyl or OH with the proviso that at least one of R₁ or R₂ is OH;
n=1-10; and
the fused ring attached to the phenol ring in Structure V is optional and when present may be aromatic, cycloaliphatic, alicyclic or heterocyclic.

The catalyst may be selected from imidazoles, substituted imidazoles, latent imidazoles, encapsulated imidazoles, phenol functionalized imidazoles, and naphthol functionalized imidazoles, as well as amidine and guanidine typed of catalysts or phenol functionalized imidazole catalysts.

The benefits and advantages of the present disclosure will become more readily apparent from the Detailed Description that follows.

### DETAILED DESCRIPTION

As noted, the disclosure provides a flux-compatible epoxy-phenol adhesive composition usual for low gap underfill applications. The composition broadly comprises an epoxy component, a phenolic component, and a catalyst.

The epoxy component may be selected from epoxy compounds having a cycloaliphatic, alicyclic, mixed cycloaliphatic-aromatic, or mixed alicyclic-aromatic backbone. Particularly useful epoxy resins are EP4088S, Eponex1510, HP7200, Hyloxy modifier 107 and mixtures thereof, shown in the formulas below, although other resins having a cycloaliphatic, mixed cycloaliphatic-aromatic backbone and/or aromatic backbone may be used. Examples include monofunctional and difunctional decahydronaphthalene glycidyl ethers supplied by Sugai Chemical Industry, mono and multifunctional glycidyl ethers based on cycloaliphatic backbones such as adamantane ring structure, including mono and multifunctional decahydronaphthalene glycidyl ether, mono and multifunctional DCPD glycidyl ethers, diglycidyl ether of hydrogenated bisphenol A, mono and multifunctional adamantyl glycidyl ethers, cycloaliphatic glycidyl esters, mono and multifunctional epoxides of cyclic monoene and polyenes and mixtures thereof.

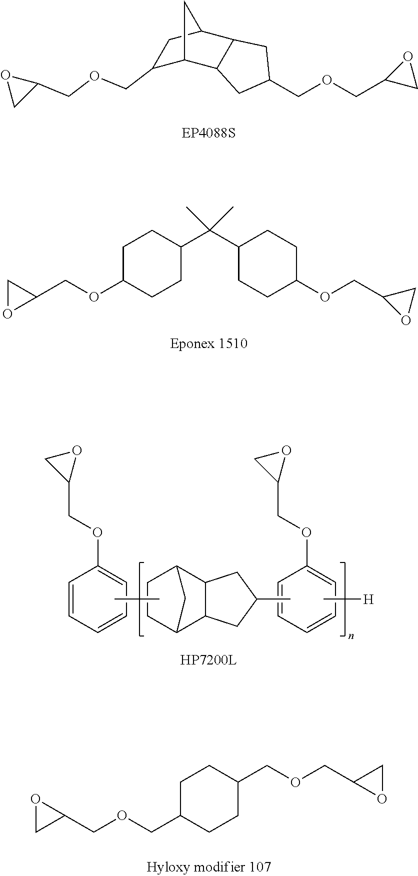

The phenolic component may be a multifunctional phenol and may be selected from those whose formulas are given below:

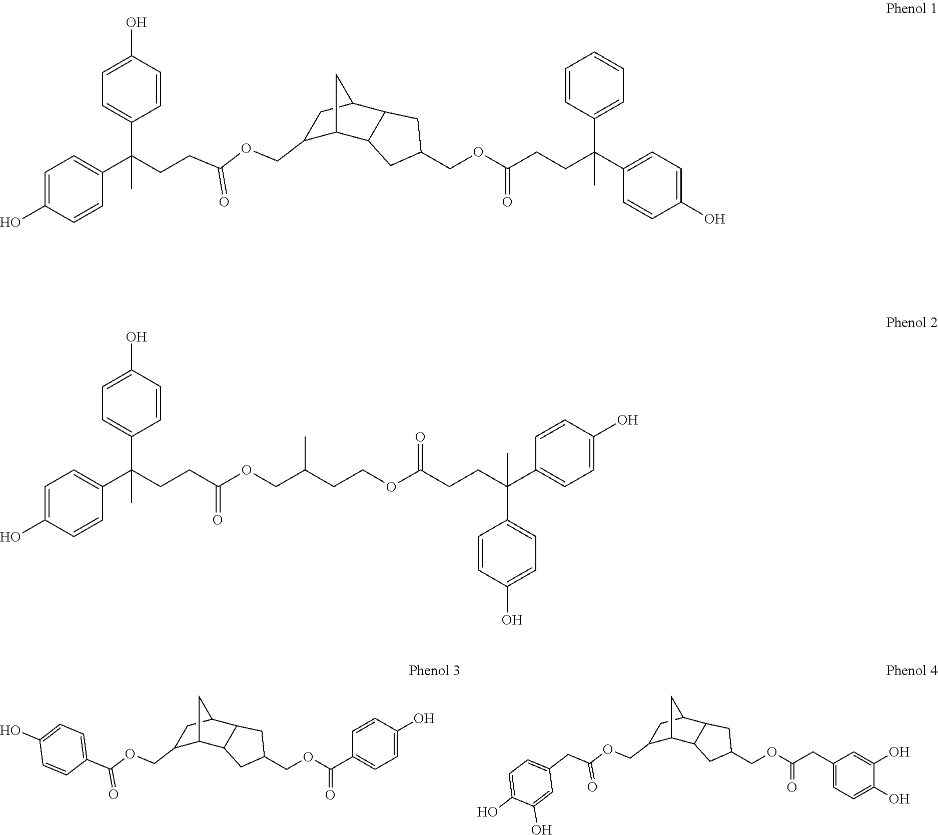 [SMILES: CC(CCC(=O)OCC1CC2C3CC(COC(=O)CCC(C)(c4ccc(O)cc4)c4ccc(O)cc4)C(C3)C2C1)(c1ccccc1)c1ccc(O)cc1 ; CC(CCOC(=O)CCC(C)(c1ccc(O)cc1)c1ccc(O)cc1)COC(=O)CCC(C)(c1ccc(O)cc1)c1ccc(O)cc1 ; O=C(OCC1CC2C3CC(COC(=O)c4ccc(O)cc4)C(C3)C2C1)c1ccc(O)cc1 ; O=C(Cc1ccc(O)c(O)c1)OCC1CC2C3CC(COC(=O)Cc4ccc(O)c(O)c4)C(C3)C2C1]

These multifunctional phenols—Phenols 1-4—are novel and form another aspect of the present disclosure.

In addition to the above novel phenols, certain known alicyclic phenols may be used as the phenolic component of the composition. Suitable phenols are sold by DIC International Chemicals under the trade name Phenolite™ phenol novolak resins. Particularly suitable known phenols are DCPD novolac and cresol novolac. Also useful are bisphenol A novolak, phenol novolak, triazine novolak, diallylbisphenol A, dihydroxynaphthalene (all isomers), 2-allylphenylnovolak, dihydroxybenzophenone (all isomers), trihydroxybenzophenone (all isomers), Rezicure 3000, bis(4-hydroxyphenyl)sulfide, and bis(4-hydroxyphenyl)sulfone.

The phenolic component may also be a multifunctional phenol represented by the general structures I, II, III and/or IV:

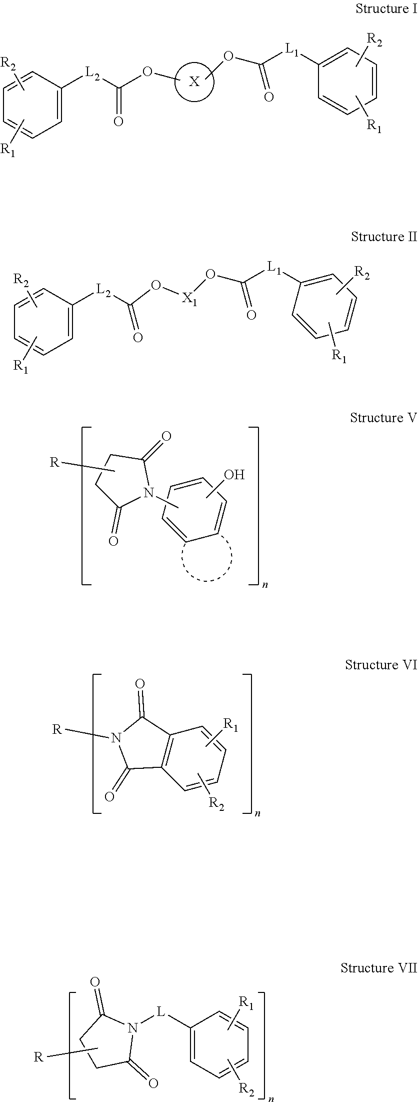

wherein R is an aliphatic, cycloaliphatic, alicyclic, mixed aromatic-cycloaliphatic or polymer backbone; in addition, R can be a fused ring in Structures V and VII;
R₁ and R₂ are H, alkyl or OH with the proviso that at least one of R₁ or R₂ is OH;
n=1-10; and
the fused ring attached to the phenol ring in Structure V is optional and when present may be aromatic, cycloaliphatic, alicyclic or heterocyclic.

The above imide or phthaleimide functional phenols can be obtained by imidization of aliphatic, alicyclic, aromatic, aralkyl amines with mono or multifunctional anhydrides. The anhydrides can be selected from methylhexahydrophthalic anhydride, nadic anhydride (methyl-5-norbornene-2,3-dicarboxylic anhydride; “MNA”) or 5-norbornene-2,3-dicarboxylic anhydride, hexahydro-4-methylphthalic anhydride (MHHPA), methyltetrahydrophthalic anhydride (MTHPA), methylcyclohexene-1,2-dicarboxylic anhydride, methylbicyclo[2.2.1] heptane-2,3-dicarboxylic anhydride, bicyclo[2.2.1] heptane-2,3-dicarboxylic anhydride, (2-dodecen-1-yl)succinic anhydride, glutaric anhydride, citraconic anhydride, methylsuccinic anhydride, 2,2-dimethylsuccinic anhydride, 2,2-dimethylgiutaric anhydride, 3-methylglutaric anhydride, 3,3-tetramethyleneglutaric anhydride, 3,3-dimethylglutaric anhydride, several isomers of hydroxyphthaleic anhydride or mixtures thereof.

The polyfunctional anhydrides that can be used for the imidization reaction include polypropylene-graft-maleic anhydride, polyethylene-graft-maleic anhydride, butadiene-

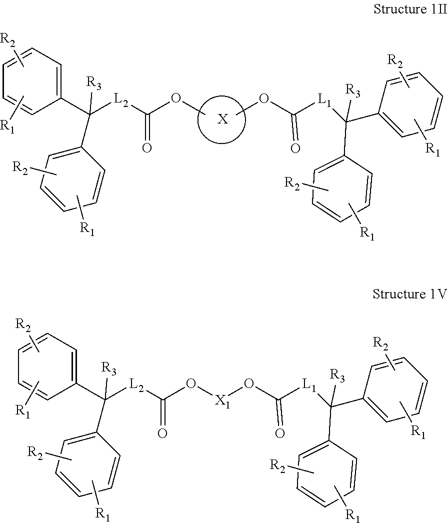

wherein X is a monocyclic, bicyclic or polycyclic ring structure that is cycloaliphatic or alicyclic optionally with aliphatic side chains; and the oxygen of the ester group is connected directly to the ring or to the side chain;
X₁ is alkylene or branched alkylene optionally comprising heteroatoms O or S;
R₃ is H, alkyl or cycloalkyl;
L₁ and L₂ are independently selected from a covalent bond, alkylene, branched alkylene, and cycloalkylene optionally comprising heteroatoms O or S;
R₁ and R₂ are independently H, methyl or OH with the proviso that at least one of R₁ or R₂ in each ring is OH; and
the ester functionality present in structures I, II, III and IV can be primary or secondary ester group.

The phenol used in the flux compatible epoxy-phenol adhesive compounds may be selected from compounds of structures V, VI and/or VII:

maleic anhydride copolymers, styrene-maleic anhydride copolymers and other copolymers and terpolymers of maleic anhydride, itaconic anhydride and citraconic anhydride.

Amines and amine functional phenols that can be used for the imidization reaction include but not limited to several isomers of aminophenol, catechol amines, aminonaphthols, dimer diamine, TCD-diamine (3(4),8(9)-bis(aminomethyl)-tricyclodecane), cyclohexylamines, aliphatic, cycloaliphatic and alicyclic primary diamines.

The epoxy-phenol adhesive compositions may further comprise a maleimide resin, which can be a bismaleimide, a polyfunctional maleimide or phenol functional maleimide of structures VIII and IX represented below.

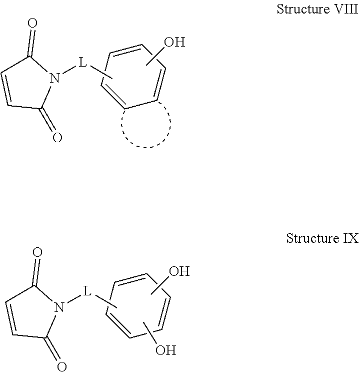

wherein L is selected from a covalent bond, alkylene, cycloalkylene, and branched alkylene optionally with hetero atoms O or S; L can also contain an ester or carbonate linkages; and

the fused ring in structure VIII is optional and when present it is aromatic, cycloaliphatic, alicyclic or heterocyclic.

The maleimide resin may be obtained by the imidization reaction of mono or multifunctional primary amines with maleic anhydride or may be obtained by Fisher esterification of mono or multifunctional aliphatic, cycloaliphatic, alicyclic or aralkyl alcohols with 6-maleimidocaproic acid. The phenol functional maleimides also may be obtained by the imidization reaction of several isomers of amino phenols, aminonaphthols, catechol amines or side chain amine functional phenols with maleic anhydride.

A variety of catalysts may be used, included among which are imidazoles, substituted imidazoles, latent imidazoles, encapsulated imidazoles, phenol functionalized imidazoles, and naphthol functionalized imidazoles. The imidazole catalyst Technicure EMI-24CN was found to be a particularly desirable curing agent. For instance, at a 4% concentration, an epoxy-phenol adhesive composition having this catalyst showed an excellent balance of Tg and other performance properties. Latent imidazoles sold under the trade name Curezol that are available from Evonik Corporation, encapsulated imidazoles from A&C catalysts and phenol or naphthol functionalized imidazoles such as Aradur 3123 can be used. Preferred catalysts include Technicure EMI 24-CN, Curezol 2-PHZ-S, Curezol 2-PZ, Curezol 2PZ-azine, Aardur 3123, and amine and polyamine functional imidazoles.

The ratio of the epoxy component to the phenolic component may be from 1:1 to 1:0.05. The ratio is preferably 1:0.2, and more preferably 1:0.1. The combination of the epoxy component and the phenolic component typically makes up about 50% of the adhesive composition, the balance being selected from curing agents, accelerators, catalysts, flow modifiers, fillers, adhesion promoters, and thixotropic agents.

In certain embodiments, the adhesive compositions may further comprise one or more flow additives, adhesion promoters, conductivity additives, rheology modifiers, or the like, as well as mixtures of any two or more thereof. Various additives may be contained in the composition as desired, for example, organic or inorganic fillers, thixotropic agents, silane coupling agents, diluents, modifiers, coloring agents such as pigments and dyes, surfactants, preservatives, stabilizers, plasticizers, lubricants, defoamers, leveling agents and the like; however it is not limited to these. In particular, the composition preferably comprises an additive selected from the group consisting of organic or inorganic filler, a thixotropic agent, and a silane coupling agent. These additives may be present in amounts of about 0.1% to about 50% by weight of the total composition, more preferably from about 2% to about 10% by weight of the total composition.

The thixotropic agent may include, but is not limited to, talc, fume silica, superfine surface-treated calcium carbonate, fine particle alumina, plate-like alumina; layered compounds such as montmorillonite, spicular compounds such as aluminum borate whisker, and the like. Among them, talc, fume silica and fine alumina are particularly desired. These agents may be present in amounts of about 1% to about 50%, more preferably from about 1% to about 30% by weight of the total composition.

The silane coupling agent may include, but is not limited to, γ-aminopropyltriethoxysilane, γ-mercaptopropyltrimethoxysilane, γ-methacryloxypropyltrimethoxysilane, γ-glycidoxypropyltrimethoxylsilane, and the like.

As used herein, “flow additives” refers to silicon polymers, ethyl acrylate/2-ethylhexyl acrylate copolymers, alkylol ammonium salt of phosphoric acid esters of ketoxime, and the like, as well as combination. Several of these additives are available from commercial sources such as BYK and Evonik Corporation.

The present disclosure provides the following non-limiting and non-exhaustive examples.

### EXAMPLES

Several imidazole catalysts were screened in phenol-epoxy formulations as shown in Table 1. Some of the formulations tested showed higher Tg when suitable imidazole accelerators are used. A liquid imidazole catalyst (Technicure EMI-24CN) appeared to perform best in terms lowering cure temperature. Low viscosity of this catalyst was an added advantage.

TABLE 1
Initial formulation screening of phenol cured epoxy system

| | | DSC | |
|---|---|---|---|
| Formu- | | peak | |
| lation | Components | temp | Tg |
| F1 | BPF DGE, 1,1,1(trishydroxyphenyl)ethane, | 125° C. | 132° C. |
| | curezol 2PZ (6%) | | |
| F2 | BPF DGE, 1,1,1(trishydroxyphenyl)ethane, | 125° C. | 115° C. |
| | 1-(2-cyanoethyl)-2-phenylimidazole | | |
| | (Technicure EMI-24CN) (5%) | | |
| F3 | 830CRP, GY 9820, | 160° C. | 138° C. |
| | 1,1,1(trishydroxyphenyl)ethane, curezol | | |
| | 2PHZ-S (5%) | | |
| F4 | 830CRP, GY 9820, | 130° C. | 126° C. |
| | 1,1,1(trishydroxyphenyl)ethane, curezol | | |
| | 2PZ (5%) | | |
| F5 | 830CRP, GY 9820, | 130° C. | 143° C. |
| | 1,1,1(trishydroxyphenyl)ethane, curezol | | |
| | 2PZ azine (5%) | | |

It was found that epoxy resins possessing a cycloaliphatic backbone showed good flux compatibility. Some of the epoxy resins that were screened for flux compatibility in neat form include EP4088S, Eponex1510, HP7200L, Hyloxy modifier 107 and a mixture of the above. All of these epoxy resins have cycloaliphatic or mixed cycloaliphatic-aromatic backbones. The flux compatibility study was performed using epoxy resins containing about 5% of flux and heating the mixture to about 80° C. for about 30 minutes and speed mixing the mixture. Upon cooling to room temperature and storing they resulted in clear mixtures without any haze.

For the phenolic component, several multifunctional phenols were made that contained cycloaliphatic or aliphatic backbones, as described in the examples below.

### Example 1: Synthesis of tetrafunctional Phenol 1

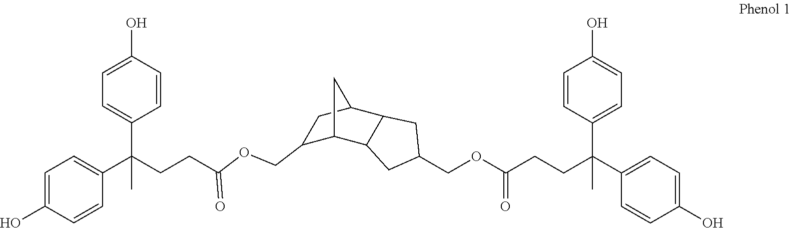 [SMILES: CC(CCC(=O)OCC1CC2C3CC(COC(=O)CCC(C)(c4ccc(O)cc4)c4ccc(O)cc4)C(C3)C2C1)(c1ccc(O)cc1)c1ccc(O)cc1]

In a 1 L 3 necked flask equipped with a thermocouple, mechanical stirrer and a condenser were placed 4,8-Bis(hydroxymethyl)tricyclo[5.2.1.0^{2,6}]decane (49.2 g, 250 mmol), diphenolic acid (143.5 g, 500 mmol), PTSA (3 g, 1.5%) in toluene 600 mL. The mixture was stirred with azeotropic distillation of water for 12h. After cooling to ambient temperature, the toluene was decanted and the remaining solid was dissolved in 1 L of ethyl acetate. The solution was washed once with water, twice with aqueous sodium bicarbonate solution and once with water. After the solution was dried over anhydrous Na₂SO₄, the solvent was evaporated using a rotary evaporator under reduced pressure. The last traces of solvent were removed under high vacuum at 80° C. for several hours to give Phenol 1 as a violet solid (152 g, 83%).

### Example 2: Synthesis of Tetrafunctional Phenol 2

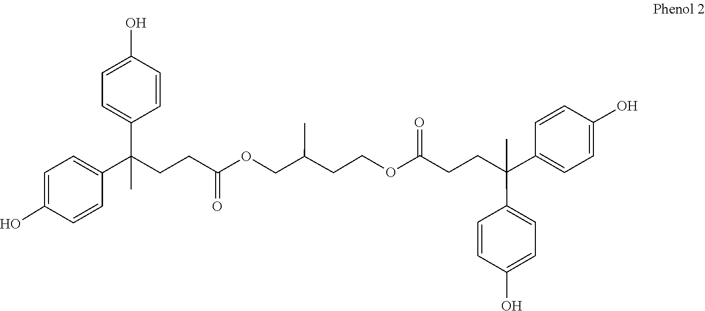 [SMILES: CC(CCOC(=O)CCC(C)(c1ccc(O)cc1)c1ccc(O)cc1)COC(=O)CCC(C)(c1ccc(O)cc1)c1ccc(O)cc1]

In a 1 L 3 necked flask equipped with a thermocouple, mechanical stirrer, and a condenser were placed 3-methyl-1,5-pentanediol (8.94 g, 75 mmol), diphenolic acid (43.11 g, 150 mmol), PTSA (1.1 g, 2.2%) in toluene 400 mL. The mixture was stirred with azeotropic distillation of water for 12h. After cooling to ambient temperature, the toluene was decanted and the resulting solid was dissolved in 600 mL of ethyl acetate. The solution was washed once with water, twice with aqueous sodium bicarbonate solution and once with water. After drying the solution over anhydrous Na₂SO₄, the solvent was evaporated using rotary evaporator under reduced pressure. The last traces of solvent were removed under high vacuum at 80° C. for several hours to give Phenol 2 as a violet solid (38 g, 88%).

### Example 3: Synthesis of Difunctional Phenol 3

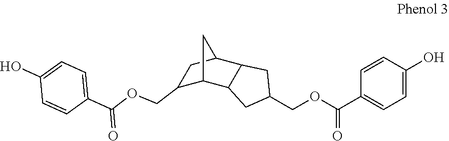 [SMILES: O=C(OCC1CC2C3CC(COC(=O)c4ccc(O)cc4)C(C3)C2C1)c1ccc(O)cc1]

In a 1 L 3 necked flask equipped with a thermocouple, mechanical stirrer and a condenser were placed 4,8-Bis(hydroxymethyl)tricyclo[5.2.1.0^{2,6}]decane (54.4 g, 277 mmol), 4-hydroxybenzoic acid (76.55 g, 554 mmol), PTSA (2.6 g, 2%) in toluene 600 mL. The mixture was stirred with azeotropic distillation of water for 12h. After cooling to ambient temperature, the toluene was decanted and the resulting solid was dissolved in 1 L of ethyl acetate. The solution was washed once with water, twice with aq. sodium bicarbonate solution and once with water. After drying the solution over anhydrous Na₂SO₄, the solvent was evaporated using rotary evaporator under reduced pressure. The last traces of solvent were removed under high vacuum at 80° C. for several hours to give phenol 3 as a violet solid (95 g, 77%).

### Example 4: Synthesis of Tetrafunctional Phenol 4

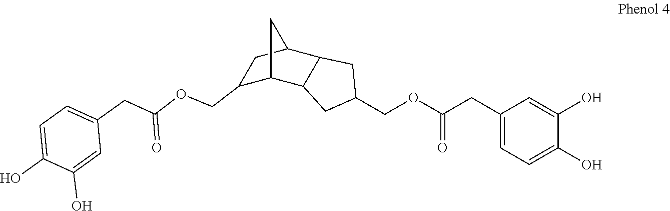 [SMILES: O=C(Cc1ccc(O)c(O)c1)OCC1CC2C3CC(COC(=O)Cc4ccc(O)c(O)c4)C(C3)C2C1]

In a 1 L 3 necked flask equipped with a thermocouple, mechanical stirrer and a condenser were placed 4,8-Bis(hydroxymethyl)tricyclo[5.2.1.0^{2,6}]decane (35 g, 177 mmol), 3,4-dihydroxyphenylacetic acid (59.96 g, 356 mmol), PTSA (1.9 g, 2%) in toluene 600 mL. The mixture was stirred with azeotropic distillation of water for 48h. After cooling to ambient temperature, the toluene was decanted and the remaining solid was dissolved in 1 L of ethyl acetate. The solution was washed once with water, twice with aqueous sodium bicarbonate solution and once with water. After drying the solution over anhydrous Na₂SO₄, the solvent was evaporated using rotary evaporator under reduced pressure. The last traces of solvent were removed under high vacuum at 80° C. for several hours to give phenol 4 as a brown solid (79 g, 82%).

Several unfilled epoxy-phenol formulations were made and screened as shown in Table 2 below.

TABLE 2
Unfilled epoxy-phenolic formulations

| | Formulations | | | | | |
|---|---|---|---|---|---|---|
| | 1 | 2 | 3 | 4 | 5 | 6 |
| | 183C | 180C | 180D | 178B | 178F | 178D |
| | (g) | (g) | (g) | (g) | (g) | (g) |
| EPN 9820 | | 5.73 | 5.44 | 3.28 | 2.66 | 1.68 |
| EP4088S | 3.73 | 1.99 | 1.89 | 3.28 | 3.04 | 1.93 |
| BPA DGE | | 3.14 | 2.98 | | | |
| HP7200L | 2.32 | | | | | |
| MY 0510 | 5.13 | | 3.24 | 7.56 | 5.48 | 3.46 |
| Hyloxy modifier 107 | | 1.47 | | | | |
| o,o′-Diallyl bisphenol A | | | | 1.414 | 0.56 | 0.707 |
| TD2131 | 1.13 | 2.0 | 1.9 | | | |
| Phenol 1 of example 1 | | | | | 0.56 | |
| Technicure EMI-24CN | 0.492 | 0.57 | 0.62 | 0.62 | 0.49 | 0.311 |
| DSC peak temp(° C.) | 140° C. | 138° C. | NA | 136° C. | 137° C. | 140° C. |
| Tg (° C.) | 154° C. | 115° C. | 155° C. | 150° C. | 144° C. | 134° C. |

Table 2 above shows several epoxy-phenolic unfilled formulations and their cure and Tg profile. The amount of imidazole catalyst was kept constant at 4 wt % in all of these the formulations. Use of Hyloxy modifier 107 was found to be beneficial for lower viscosity. However, this cycloaliphatic epoxy negatively affected the Tg. Use of tetrafunctional phenol 1 appeared to increase the Tg significantly as compared to the formulation that used diallylbisphenol A (Formulation 5 vs 6 in Table 2).

Since cycloaliphatic epoxy resins showed good flux compatibility in neat form, several filled formulations were made blending them with other epoxy resins to get a balance of Tg, viscosity and flux compatibility. Several epoxy-phenolic formulations were developed that at least partially contained epoxy resins possessing cycloaliphatic backbone to improve flux compatibility. In contrast to unfilled formulations, filled formulations showed significant decrease in Tg when silica was used as filler as compared to the equivalent unfilled formulations. To obtain a Tg in the range of 120-140° C., the formulations were modified by adding multifunctional phenols possessing cycloaliphatic-aliphatic backbones to make the formulations shown in Table 3.

TABLE 3
Epoxy-phenolic filled formulations using flux compatible epoxy resins

| | Formulations | | | | | |
|---|---|---|---|---|---|---|
| | 1 | 2 | 3 | 4 | 5 | 6 |
| | (g) | (g) | (g) | (g) | (g) | (g) |
| | 178D | 178F | 180D | 180E | 183C | 183D |
| EPN 9820 | 10.06 | 10.04 | 16.21 | 12.95 | | |
| EP4088S | 11.313 | 11.30 | 5.63 | 4.49 | 13.97 | 11.98 |
| BPA DGE | | | 8.89 | 7.1 | | |
| HP7200L | | | | | 8.68 | 7.45 |
| MY0510 | 20.531 | 20.51 | 9.662 | 17.0 | 19.24 | 23.065 |
| Tetrafunctional phenol 1 | | 2.13 | | | | |
| Phenol-novolac TD2131 | | | 5.72 | 4.57 | 4.24 | 3.62 |
| O,O′-diallylbisphenol A | 4.23 | 2.13 | | | | |
| Technicure EMI-24CN | 1.86 | 1.87 | 1.85 | 1.85 | 1.85 | 1.85 |
| SE2050 silica | 50.0 | 50.0 | 49.975 | 49.975 | 49.975 | 49.975 |
| KD1 | 1.0 | 1.0 | 1.0 | 1.0 | 1.0 | 1.0 |
| W9010 | 0.5 | 0.5 | 0.5 | 0.5 | 0.5 | 0.50 |
| Z6040 | 0.5 | 0.5 | 0.5 | 0.5 | 0.5 | 0.50 |
| PC1344 | 0.05 | 0.05 | 0.05 | 0.05 | 0.05 | 0.05 |
| Total | 100.0 | 100.0 | 100.0 | 100.0 | 100.0 | 100.0 |

The Tg and viscosity profiles of several of the filled formulations of Table 3 are shown in Table 4. One distinct feature of the epoxy-phenolic chemistry was an increase in Tg observed after the 2^{nd} DSC Tg ramp even though the DSC peak temperature was lower. This result may be coming from additional crosslinking during the 2^{nd} heating. The increased Tg might benefit in the reliability of the device when it is subjected to multiple solder reflow conditions.

TABLE 4
Cure profile, viscosity and Tg of prototype formulations

| | DSC Peak Cure | Viscosity | 1^{st} Tg | 2^{nd} Tg |
|---|---|---|---|---|
| | Temperature | @ 20 s⁻¹ | after | after |
| Formu- | (° C.) | (cP) | 25-260° C. | 2^{nd} ramp of |
| lation | w/o flux | 0 hr | DSC ramp | 25-260° C. |
| 1 | 160 | 3169 | 99 | 119 |
| 2 | 150 | 7377 | 113 | 128 |
| 3 | 139 | 48902 | 123 | 127 |
| 4 | 137 | 23291 | 141 | 143 |
| 5 | 138 | 16441 | 120 | 132 |
| 6 | 138 | 10972 | 126 | 143 |

What is claimed is:

1. A flux-compatible epoxy-phenol adhesive for low-gap underfill applications comprising:
an epoxy component comprising an epoxy compound having a cycloaliphatic, alicyclic or mixed cycloaliphatic-aromatic, alicyclic-aromatic backbone;
a multifunctional phenolic component; and
a catalyst.

2. The flux-compatible epoxy-phenol adhesive of claim 1, wherein the epoxy compound is selected from EP4088S, Eponex1510, HP7200L, Hyloxy modifier 107, mono and multifunctional decahydronaphthalene glycidyl ether, mono and multifunctional DCPD glycidyl ethers, diglycidyl ether of hydrogenated bisphenol A, mono and multifunctional adamantyl glycidyl ethers, cycloaliphatic glycidyl esters, glycidyl compounds derived from cycloaliphatic monoamines and diamines, mono and multifunctional epoxides of cyclic monoene and polyenes and mixtures thereof.

3. The flux-compatible epoxy-phenol adhesive of claim 1, wherein the catalyst is selected from imidazoles, substituted imidazoles, latent imidazoles, encapsulated imidazoles, phenol functionalized imidazoles, and naphthol functionalized imidazoles.

4. The flux-compatible epoxy-phenol adhesive of claim 3, wherein the catalyst is selected from Technicure EMI 24-CNI, Curezol 2-PHZ-S, Curezol 2-PZ, Curezol 2PZ-azine, ikardar 3123, Ajicure series, Technicure series, Resicure series, Technirez series, and amine and polyamine functional imidazoles.

5. The flux-compatible epoxy-phenol adhesive of claim 1, wherein the phenol is selected from structures I, II, III and/or IV: 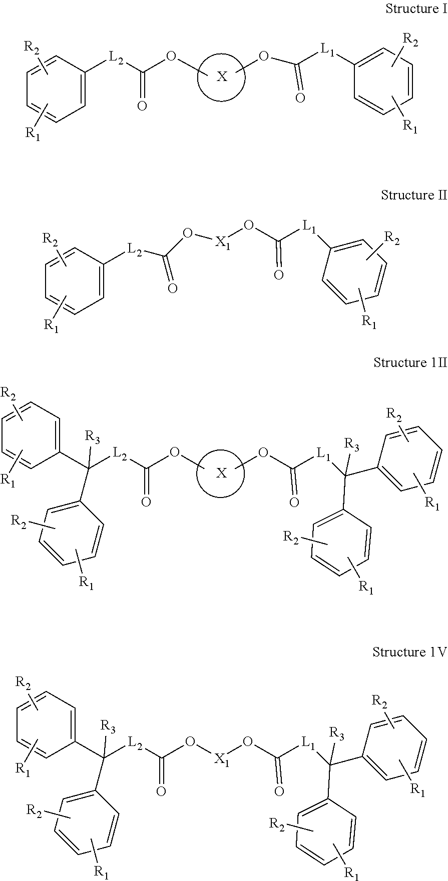
wherein X is a monocyclic, bicyclic or polycyclic ring structure that is cycloaliphatic or alicyclic optionally with aliphatic side chains; the oxygen of the ester group is connected directly to the ring or to the aliphatic side chain;
X₁ is alkylene or branched alkylene optionally comprising hetero atoms O or S;
R₃ is H, alkyl or cycloalkyl;
L₁ and L₂ are independently selected from a covalent bond, alkylene, branched alkylene, and cycloalkylene optionally comprising heteroatoms O or S;
R₁ and R₂ are independently H, methyl or OH with the proviso that at least one of R₁ or R₂ in each ring is OH; and
the ester group present in structures I, II, III and IV can be primary or secondary ester group.

6. The flux-compatible epoxy-phenol adhesive of claim 1, wherein the multi-functional phenolic component is selected from the following phenolic compounds: 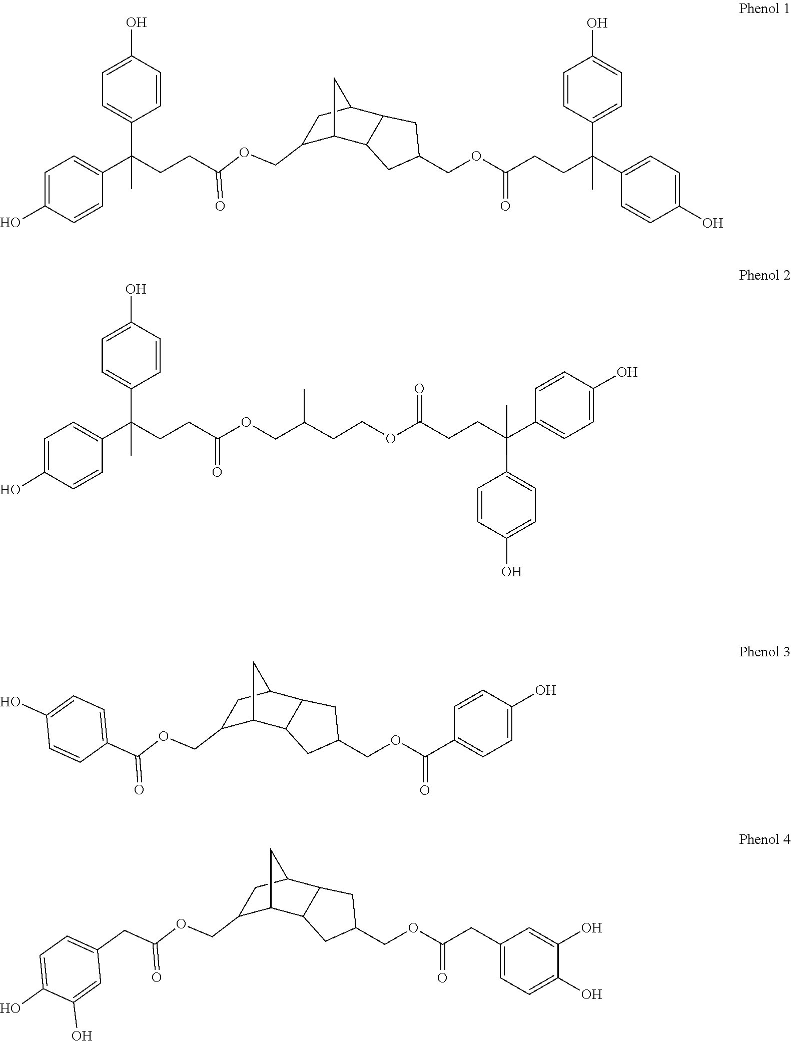 [SMILES: CC(CCC(=O)OCC1CC2C3CC(COC(=O)CCC(C)(c4ccc(O)cc4)c4ccc(O)cc4)C(C3)C2C1)(c1ccc(O)cc1)c1ccc(O)cc1 ; CC(CCOC(=O)CCC(C)(c1ccc(O)cc1)c1ccc(O)cc1)COC(=O)CCC(C)(c1ccc(O)cc1)c1ccc(O)cc1 ; O=C(OCC1CC2C3CC(COC(=O)c4ccc(O)cc4)C(C3)C2C1)c1ccc(O)cc1 ; O=C(Cc1ccc(O)c(O)c1)OCC1CC2C3CC(COC(=O)Cc4ccc(O)c(O)c4)C(C3)C2C1]
DCPD novolak, cresol novolak, bisphenol A novolak, phenol novolak, triazine novolak, diallylbisphenol A, dihydroxynaphthalene (all isomers), 2-allylphenylnovolak, dihydroxybenzophenone (all isomers), trihydroxybenzophenone (all isomers), Rezicure 3000, bis(4-hydroxyphenyl)sulfide, and bis(4-hydroxyphenyl)sulfone.

7. The flux-compatible epoxy-phenol adhesive of claim 1, wherein the phenolic component is selected from structures V, VI and/or VIII: 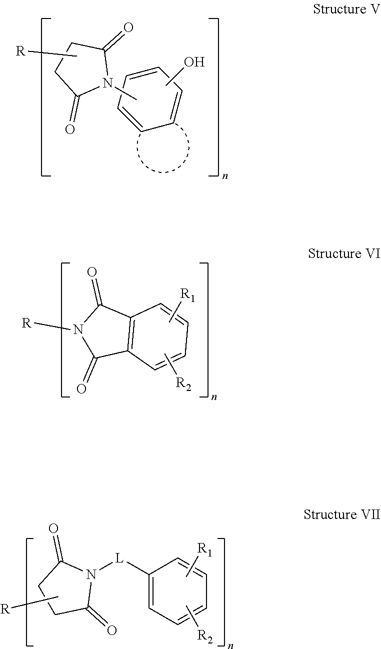
wherein R is an aliphatic, cycloaliphatic, alicyclic, mixed aromatic-cycloaliphatic or polymer backbone; in addition, R can be a fused ring in Structures V and VII;
R₁ and R₂ are H, alkyl or OH with the proviso that at least one of R₁ or R₂ is OH;
n=1-10; and
the fused ring attached to the phenol ring in Structure V is optional and when present may be aromatic, cycloaliphatic, alicyclic or heterocyclic.

8. The flux-compatible epoxy-phenol adhesive of claim 1 further comprising a maleimide resin.

9. The flux-compatible epoxy-phenol adhesive of claim 8 wherein the maleimide resin is a bismaleimide, a polyfunctional maleimide of structures VIII and IX: 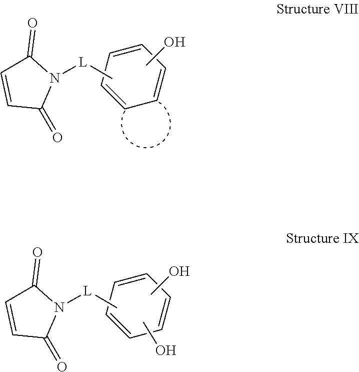
wherein L is selected from a covalent bond, alkylene, cycloalkylene, and branched alkylene optionally with hetero atoms O or S; L can also contain an ester or carbonate linkages; and
the fused ring in structure VIII is optional and when present it is aromatic, cycloaliphatic, alicyclic or heterocyclic.

10. The flux-compatible epoxy-phenol adhesive of claim 9 wherein the bismaleimide or polyfunctional maleimide is obtained by imidization of mono or multifunctional primary amines with maleic anhydride or obtained by Fisher esterification of mono or multifunctional aliphatic, cycloaliphatic, alicyclic or aralkyl alcohols with 6-maleimidocaproic acid.

11. The flux-compatible epoxy-phenol adhesive of claim 1, wherein the ratio of the epoxy resin to the phenol is from 1:1 to 1:0.05.

12. The flux-compatible epoxy-phenol adhesive of claim 1 which further comprises curing agents, accelerators, catalysts, flow modifiers, fillers, adhesion promoters and thixotropic agents.

13. A phenol selected from:

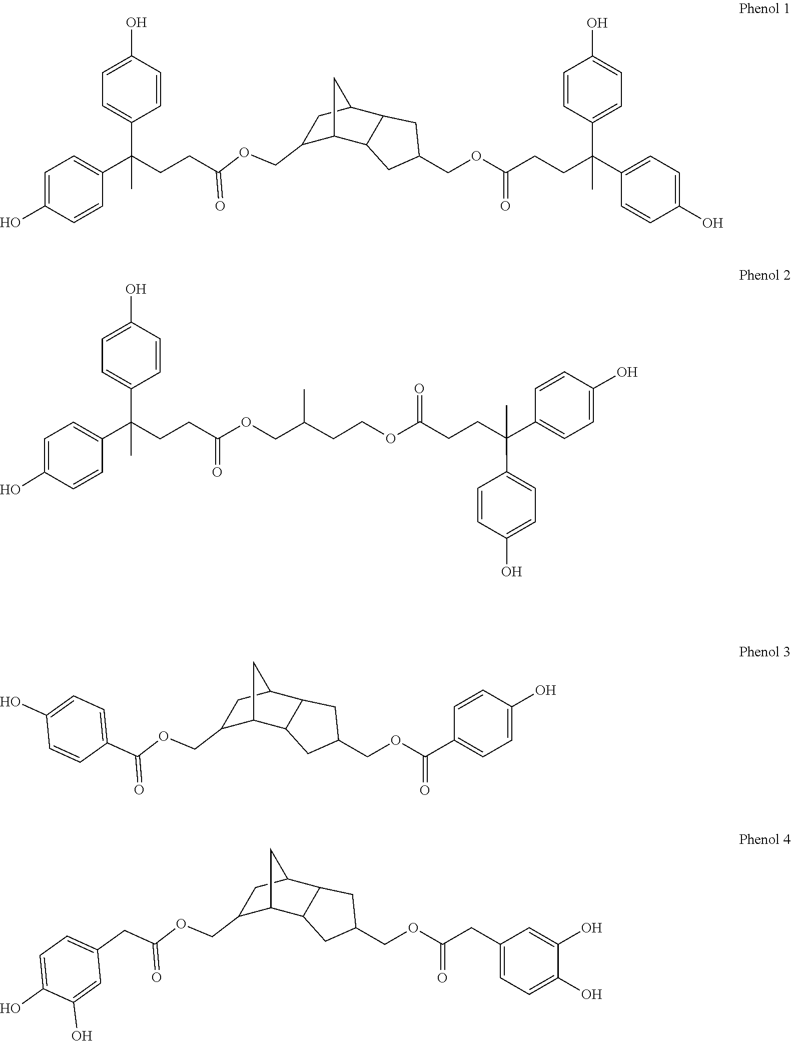 [SMILES: CC(CCC(=O)OCC1CC2C3CC(COC(=O)CCC(C)(c4ccc(O)cc4)c4ccc(O)cc4)C(C3)C2C1)(c1ccc(O)cc1)c1ccc(O)cc1 ; CC(CCOC(=O)CCC(C)(c1ccc(O)cc1)c1ccc(O)cc1)COC(=O)CCC(C)(c1ccc(O)cc1)c1ccc(O)cc1 ; O=C(OCC1CC2C3CC(COC(=O)c4ccc(O)cc4)C(C3)C2C1)c1ccc(O)cc1 ; O=C(Cc1ccc(O)c(O)c1)OCC1CC2C3CC(COC(=O)Cc4ccc(O)c(O)c4)C(C3)C2C1]

14. A phenol selected from one or more of structures I, II, III and IV: 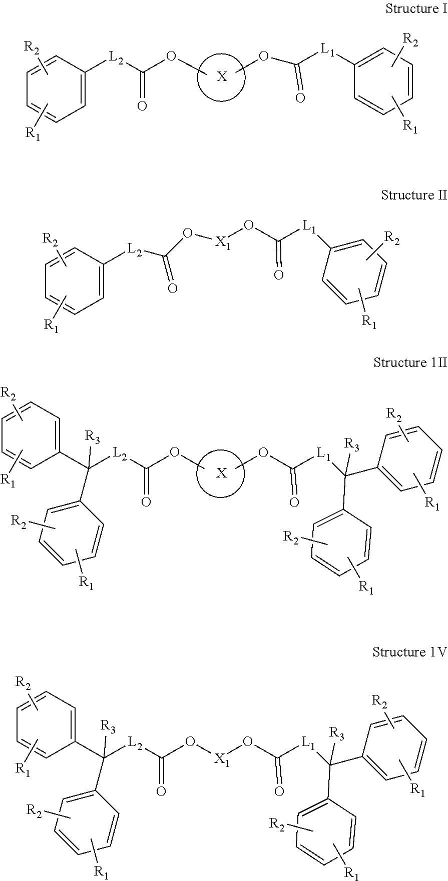
wherein X is a monocyclic, bicyclic or polycyclic ring structure that is cycloaliphatic or alicyclic optionally with aliphatic side chains; and the oxygen of the ester group is connected directly to the ring or to the aliphatic side chain;
X₁ is alkylene or branched alkylene optionally with hetero atoms O or S;
R₃ is H, alkyl or cycloalkyl;
L₁ and L₂ are independently selected from a covalent bond, alkylene, branched alkylene, and cycloalkylene optionally comprising heteroatoms O or S;
R₁ and R₂ are independently H, methyl or OH with the proviso that at least one of R₁ or R₂ in each ring is OH; and
the ester group present in structures I, II, III and IV can be a primary or secondary ester group.

15. A phenol selected from one of more of structures V, VI and VII: 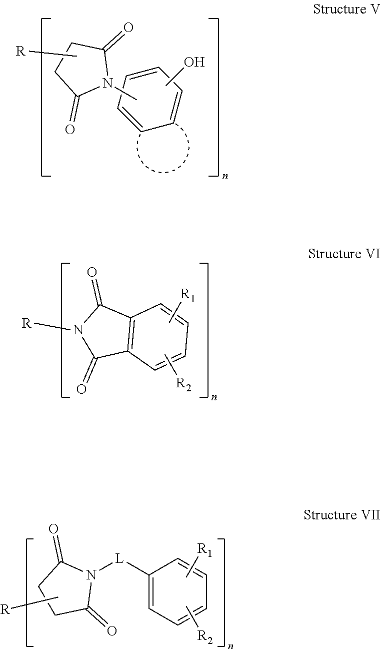
wherein R is an aliphatic, cycloaliphatic, alicyclic, mixed aromatic-cycloaliphatic or polymer backbone; in addition, R can be a fused ring in structures V and VII;
R₁ and R₂ are H, alkyl or OH with the proviso that at least one of R₁ or R₂ is OH;
n=1-10; and
the fused ring attached to the phenol ring in structure V is optional and when present may be aromatic, cycloaliphatic, alicyclic or heterocyclic.

16. A phenol selected from one or more of structures VIII and IX: 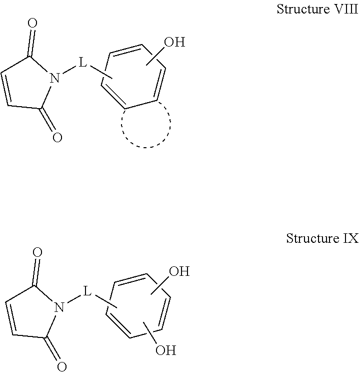
wherein L is selected from a covalent bond, alkylene, cycloalkylene, and branched alkylene optionally with hetero atoms O or S; L can also contain an ester or carbonate linkages; and
the fused ring in structure VIII is optional and when present it is aromatic, cycloaliphatic, alicyclic or heterocyclic.

17. The flux-compatible epoxy-phenol adhesive of claim 1 wherein:
the epoxy compound is selected from EP4088S, Eponex1510, HP7200L, Hylox modifier 107 and mixtures thereof;
the multifunctional phenol is selected from: 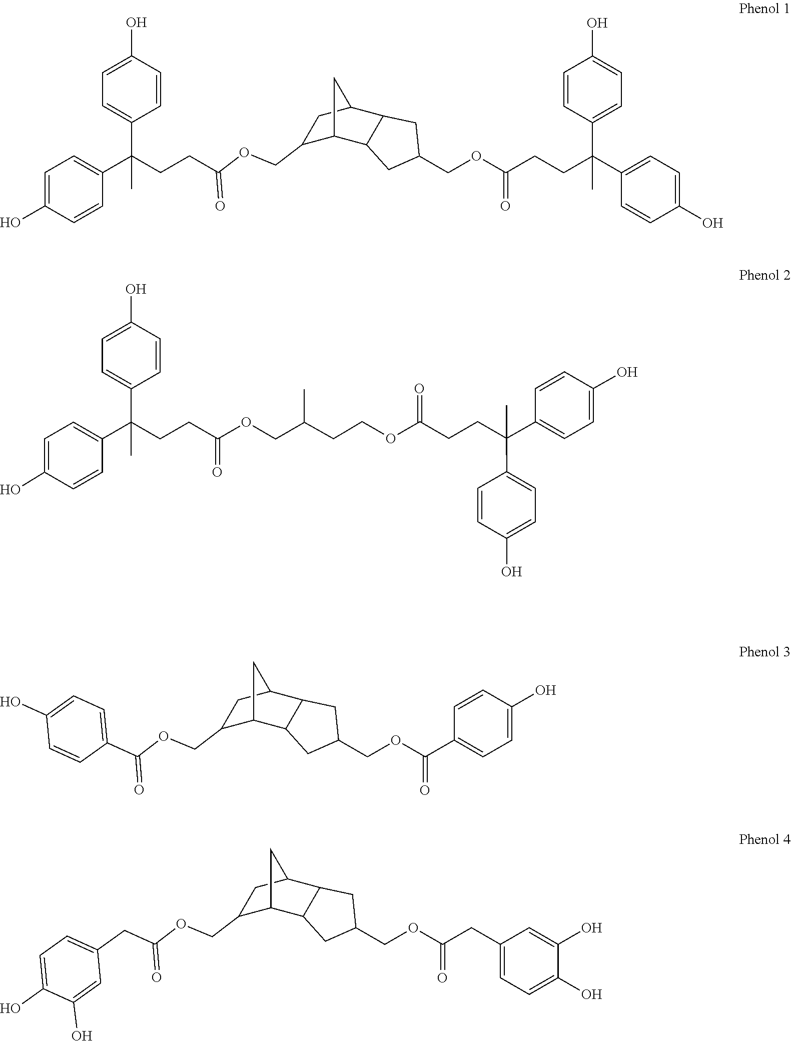 [SMILES: CC(CCC(=O)OCC1CC2C3CC(COC(=O)CCC(C)(c4ccc(O)cc4)c4ccc(O)cc4)C(C3)C2C1)(c1ccc(O)cc1)c1ccc(O)cc1 ; CC(CCOC(=O)CCC(C)(c1ccc(O)cc1)c1ccc(O)cc1)COC(=O)CCC(C)(c1ccc(O)cc1)c1ccc(O)cc1 ; O=C(OCC1CC2C3CC(COC(=O)c4ccc(O)cc4)C(C3)C2C1)c1ccc(O)cc1 ; O=C(Cc1ccc(O)c(O)c1)OCC1CC2C3CC(COC(=O)Cc4ccc(O)c(O)c4)C(C3)C2C1]
and the catalyst is selected from Technicure EMI 24-CN, Curezol 2-PHZ-S, Curezol 2-PZ, Curezol 2PZ-azine, and Aardur 3123.
